(19) United States
(12) Patent Application Publication
(10) Pub. No.: US 2015/0250478 A1
(43) Pub. Date: Sep. 10, 2015

(54) FILMS, KITS AND METHODS FOR ENHANCING TISSUE TREATMENT BY PLASMA WELDING

(71) Applicant: IONMED LTD., Yokneam Illit (IL)

(72) Inventors: Amnon Lam, Kibbutz Givat Oz (IL); Michael Maller, Eshchar (IL)

(21) Appl. No.: 14/437,227

(22) PCT Filed: Oct. 21, 2013
(86) PCT No.: PCT/IL2013/050846

§ 371 Date: Apr. 21, 2015

Related U.S. Application Data
(60) Provisional application No. 61/716,549, filed on Oct. 21, 2012.

(51) Int. Cl. A61B 17/08; A61B 18/04

(52) U.S. Cl. CPC A61B 17/085; A61B 18/042; A61B 2017/00004

(57) ABSTRACT

Films made of biocompatible material selected to enhance tissue treatment by plasma welding are provided. The films may be reinforced in various ways, adhesively attached to various tissues and participate in treating processes such as wound closure and fixation, wound healing and organ welding, supported by plasma application which enhances treatment as well as attachment of the films to the tissues. Dimensions and characteristics of the films as well as of applicator heads are adapted to optimize healing.

### BACKGROUND OF THE INVENTION

1. Technical Field

The present invention relates to the field of wound treatment, and more particularly, to wound treatment by plasma welding.

2. Discussion of Related Art

Plasma welding is an innovative wound treatment method, disclosed in WIPO documents nos. WO2011055368, WO2011055368 and WO2012153332, which are incorporated herein by reference in their entirety. Plasma application promotes wound healing and results in finer scars than other wound treatment methods.

### SUMMARY OF THE INVENTION

One aspect of the present invention provides a film made of biocompatible material selected to enhance tissue treatment by plasma welding, respective methods and kits.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1 is a high level schematic illustration of a wound in a tissue, a film and an applicator head for plasma treating wounds or tissues, according to some embodiments of the invention.

FIGS. 2 and 3 are high level schematic illustrations of reinforced films, according to some embodiments of the invention.

FIGS. 4A-4F are high level schematic illustrations of an applicator head, according to some embodiments of the invention.

FIGS. 5A-5D are high level schematic illustrations of film cross sections, according to some embodiments of the invention.

FIG. 6 is a high level schematic illustration of a method, according to some embodiments of the invention

### DETAILED DESCRIPTION OF THE INVENTION

Prior to the detailed description being set forth, it may be helpful to set forth definitions of certain terms that will be used hereinafter.

The term “tissue” as used in this application refers to any type of biological tissue, internal or external, as well as to any type of tissue lesion, such as a cut or a wound in the tissue. In case of cuts or wounds, the terms “edges” or “sides” of the cut or wound, as used in this application refer to any part of the circumference of the cut or wound or their periphery.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

Films made of biocompatible material selected to enhance tissue treatment by plasma welding are provided. The films may be reinforced in various ways, adhesively attached to tissues or wounds and participate in treating processes such as wound closure and fixation and wound healing, as well as any other tissue treatment, organ welding etc., supported by plasma application which enhances treatment as well as attachment of the films to the tissue (e.g. wound or lesion). Dimensions and characteristics of the films as well as of applicator heads are adapted to optimize healing and usability.

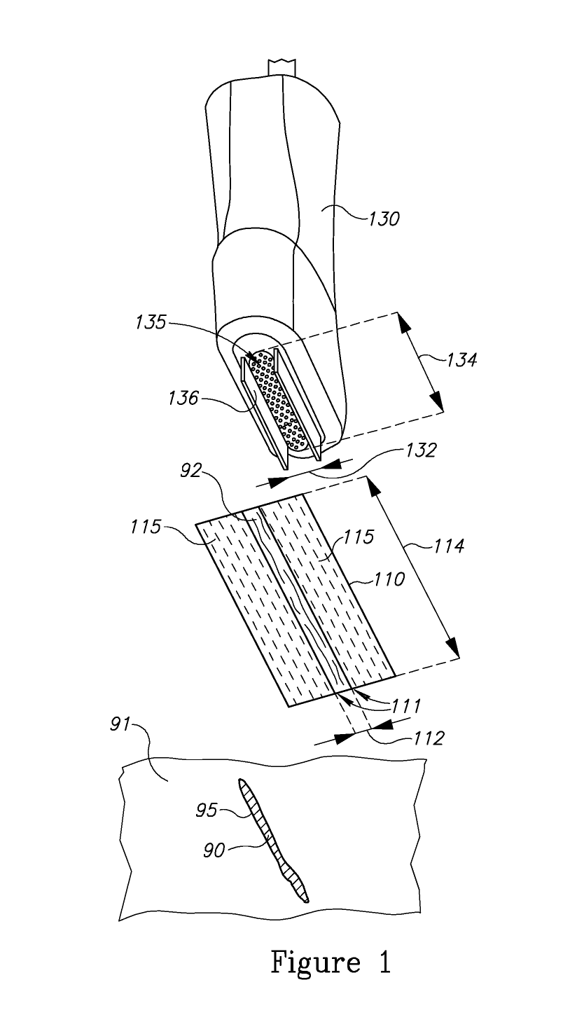

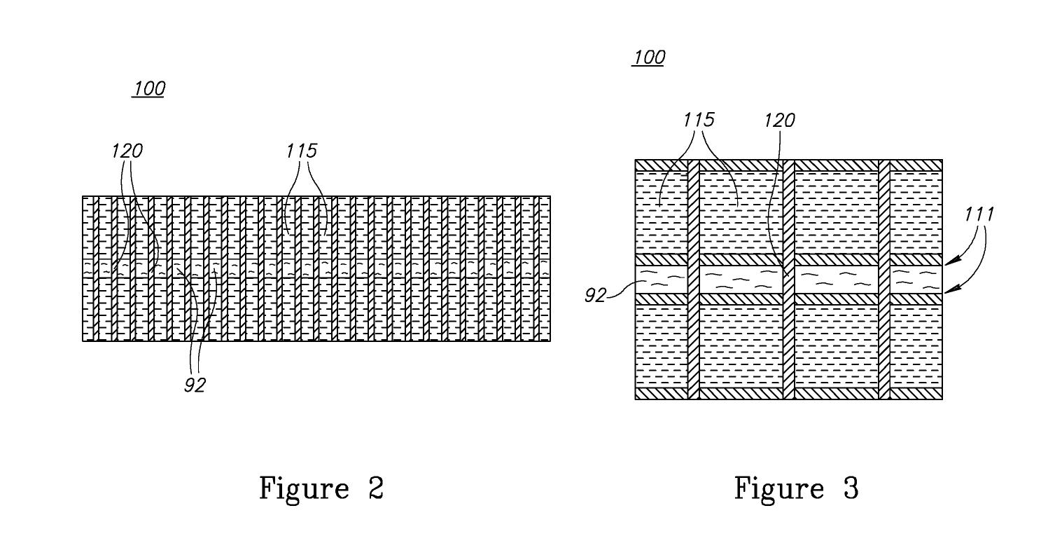

FIG. 1 is a high level schematic illustration of a wound 90 in tissue 91, a film 110 and an applicator head 130 for plasma treating wound 90 or tissue 91, according to some embodiments of the invention. FIGS. 2 and 3 are high level schematic illustrations reinforced films 110, according to some embodiments of the invention.

Film 110 is made of biocompatible material 92 selected to enhance tissue treatment by plasma welding. Biocompatible material 92 may be selected to degrade or disintegrate over time. In certain non-limiting examples, plasma welding may be carried out by cold plasma non charring plasma, e.g., at 40° C.

Film 110 may be formed as an elongated strip (FIG. 1) and further comprise attached adhesive tape 115 on at least one long side 111 of the elongated strip. In certain embodiments, adhesive tape 115 may be attached on both long sides 111 of the elongated strip (FIGS. 2, 3).

Biocompatible material 92 may comprise chitosan and may be translucent or transparent. Biocompatible material 92 may soften upon contact with tissue exudates (e.g., blood) and stick to edges 95 of wound 90. Biocompatible material 92 may be selected to promote coagulation through its mechanical, chemical and/or biological properties, in themselves and/or in combination with the plasma application.

Film 110 may comprise at least one reinforcement 120 interconnecting the attached adhesive tapes 115 on the sides of the elongated strip as illustrated in FIG. 3. In certain embodiments, reinforcements 120 may comprise bridges between adhesive tapes 115, made e.g. of medical plaster, synthetic fibers or even pre-heated biocompatible material 92 (e.g., chitosan). In certain embodiments reinforcements 120 may be 1-3 mm wide and be 3-10 mm apart.

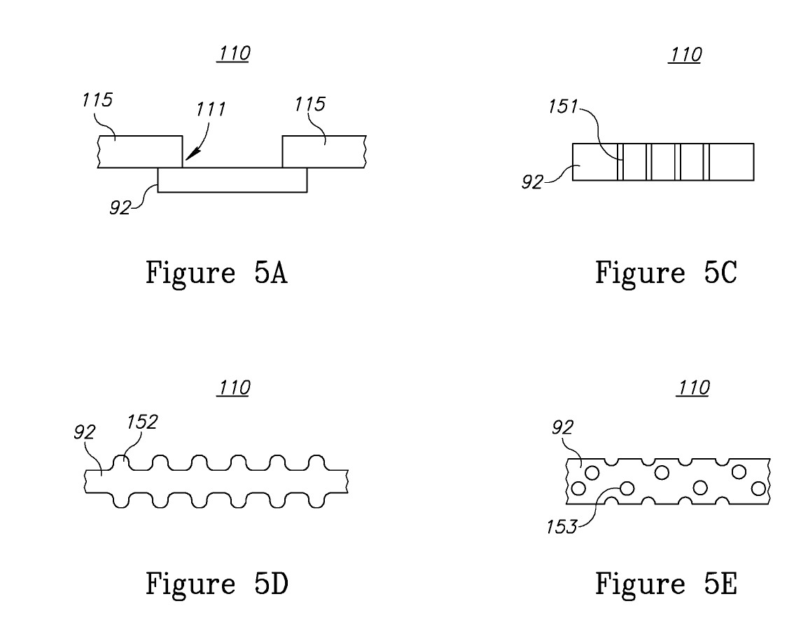

Film 110 may comprise at least one reinforced zone 120 of pre-heated film material, as illustrated in FIG. 2. For example, film 110 may comprise a plurality of linear reinforced zones 120 traversing a narrow dimension of elongated film strip 110, as illustrated in FIG. 2. In certain embodiments, linear reinforced zones 120 may be 1-3 mm wide and be 3-10 mm apart. Linear reinforced zones 120 may interconnect the attached adhesive tapes 115 on the sides of elongated film strip 110. In certain embodiments, linear reinforced zones 120 may be produced across, along, or at any other orientation on film 110. In certain embodiments, linear reinforced zones 120 may crisscross film 110 or be produced with variable orientation. Film 110 may be reinforced by embedded reinforcement fibers. A thickness of film 110 may be selected according to expected mechanical strains. Thickened zones 152 (FIG. 5B) in film 110 may be configured to enhance its mechanical strength. Thickening film material and embedding fibers in the film may be carried out to prevent tearing of film 110 during application to the tissue and/or during plasma application.

In certain embodiments, application of film 110 may be accompanied by a removable or degradable suture or staple or by additional adhesive films.

Film 110 is arranged to overcome chitosan's softening upon contact with wound fluids and blood or other tissue exudates. Reinforcements 120 are arranged to allow handling film 110 and keeping its form while maintaining the ability of film 110 to cover the wound or tissue and attach the wound's edges. Reinforcements 120 are arranged to provide sufficient mechanical support to film 110, to allow easy handling of film 110 and efficient closure of wound 90 and treatment of tissue 91 in face of possible softening of the chitosan material. In certain embodiments, pre-heating regions 120 of film 110 improve the physical properties (e.g., tensile strength and elasticity) of film 110 upon contact with the patient's blood. The chitosan material may be seen as tissue solder, and film 110 may be seen as a solder film, designed to allow soldering wound 90 by plasma welding.

In certain embodiments, film 110 comprises an adhesive wound closure that comprises a sheet of solder film, a first elongated band of adhesive on a first side of the sheet and a second elongated band of adhesive on a second side of the sheet. The first and second adhesive bands (adhesive tapes 115) bound an intermediate non-adhesive band of the solder material (biocompatible material 92) and the composite structure of the first band, the second band, and the intermediate non-adhesive band of solder material has a tensile strength chosen so that when the first elongated band of adhesive is adhered along one elongated edge of a tissue incision, and the second elongated band is adhered along a second elongated edge of a tissue incision, the composite structure holds opposing incision edges adjacent each other with the non-adhesive intermediate band overlying the opposing incision edges. Solder film 110 may be configured to allow plasma passage through it and interact with the incision below it. Solder film 110 may comprise chitosan, albumin, fibrin, and/or other natural or synthetic biocompatible material 92. In certain embodiments, adhesive tapes 115 may comprise adhesive and removable carrier material to protect the adhesive until use. In certain embodiments, film 110 may be between 7 cm and 20 cm long (114), or longer. In certain embodiments, film 110 may be at least 3 mm wide (112). In certain embodiments, adhesive tapes 115 may be attached to a wide film of biocompatible material 92 and spaced at least 3 mm apart (112). In certain embodiments, adhesive tapes 115 may comprise polyester nonwoven material that is coated with hypoallergenic, pressure sensitive acrylate adhesive and covered with a silicon liner until application to the skin.

In certain embodiments, film 110 may be part of a larger sheet configured to be cut by medical personnel before actual application, thereby allowing selection of an appropriate size of the applied film. The sheet may be perforated or otherwise pre-formed to enable easy selection of the wanted film size, or may be simply cut to the right size. The sheet or multiple sheets (possibly with cutting means) may be part of kit 100 described below.

In certain embodiments, film 110 may comprise an absorption capacity (FIG. 5D) to absorb wound or tissue fluid. In certain embodiment, film 110 may be permeable (e.g., be perforated, FIG. 5C) to allow passage of tissue exudates and thereby enhance either or both plasma transmission to wound 90 or tissue 91 and removal of wound fluid and tissue exudates.

In certain embodiments, film 110 may be cured by the plasma application and thus hardened to mechanically stabilize the treatment area. The curing may enhance adhesion to the tissue and resistance to fluids, and determine the degree of strength and permeability of the welded film.

Film 110 may further comprise an antiseptic agent and/or an antibiotic material selected to enhance tissue treatment. For examples, film 110 may be dipped or impregnated with antiseptics and/or antibiotics.

In certain embodiments, film 110 may comprise least one plasma-activated compound, e.g., selected to create free radicals upon activation by plasma. In non-limiting examples, such plasma-activated compound may comprise silver, silver salts or acetylate.

Film 110 and applicator head 130 may be provided in a kit 100, wherein film 110 is made of biocompatible material 92 selected to enhance tissue treatment by plasma welding and applicator head 130 is arranged to connect to a plasma generating device (not shown) and to plasma-treat tissue 91.

Film 110 may have a specified width 112 and applicator head 130 may have a width 132 smaller than specified width 112 of film strip 110. In certain embodiments, applicator head 130 may be at least three times longer (134) than wide (132). In certain embodiments, applicator head 130 may be at least twice longer (134) than wide (132) or may be at least five time longer than wide. The dimensions of applicator head 130 may be adapted to the type of treatment and to the type and form of film 110. In certain embodiments, applicator head 130 may be between 3 cm and 9 cm long. Applicator head 130 may be perforated to model and control the generated plasma and its uniformity.

Applicator head 130 may comprise spacers 136 configured to optimize plasma welding of wound 90 or tissue 91 and film 110. In certain embodiments, spacers 136 may be 3-9 mm apart and maintain a distance of between 4-8 mm between the electrode and film 110. In certain embodiments, applicator head 130 may comprise plastic dielectric material between an electrode and a plasma formation zone 135 enclosed by applicator head 130. In certain embodiments, the plastic dielectric material may be 0.1-3 mm thick. The inventors have discovered that these measures provide optimal operational conditions. The invention however is not limited to this choice of parameters. In certain embodiments, the electrode in applicator head 130 (illustrate) may be spiral, circular or half circular, as non-limiting examples. Applicator head 130 may be designed according to principles illustrated in the applicant's earlier disclosures, WIPO documents nos. WO2011055368, WO2011055368 and WO2012153332. Kit 100 may further comprise an exciter band (not shown) for initiating the plasma, e.g. one comprising a grooved plastic sleeve and a conductive loop in the sleeve.

In certain embodiments, film 110 and applicator head 130 are designed to operate under a gas flux across plasma formation zone 135 enclosed by applicator head 130 that is between 0.05 and 0.4 liters/min•mm², and, with respect to power supplied to an energy emitter in the plasma generating device (not shown), a duty cycle between 2.5% and 15%, a carrier frequency between 0.5 MHz and 5 MHz, and a RF voltage 2.5 kV and 7 kV. The inventors have discovered that these operational conditions are optimal. The invention however is not limited to this choice of parameters.

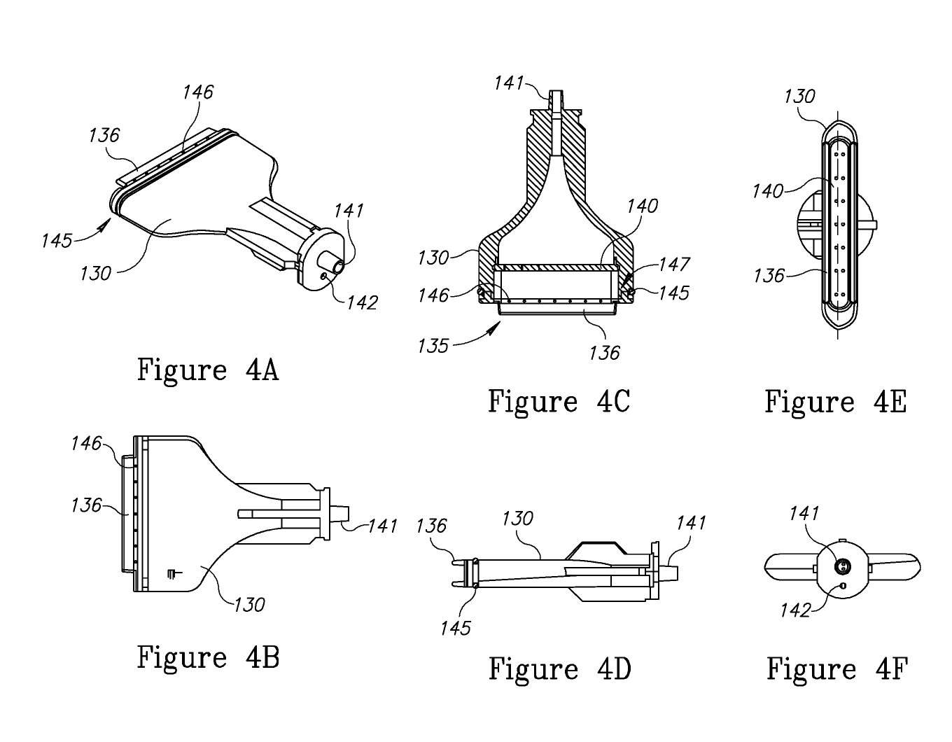

FIGS. 4A-4F are high level schematic illustrations of applicator head 130, according to some embodiments of the invention. FIG. 4A is a perspective view, FIG. 4B is a top view, FIG. 4C is a top cross section view, FIG. 4D is a side view, FIG. 4E is a front view and FIG. 4F is a back view. FIGS. 4A-4F illustrate applicator head 130 with spacer 136, optionally having pores 146 to enable gas escape from plasma formation zone 135 in case spacers 136 are in close contact with the skin or with film 110. A perforated plate 140, also termed “shower head” below, controls gas flow to be uniform and in a pre-defined flow rate to form uniform plasma (see below for more details. An electrode 145 (inserted in a groove at the periphery of applicator head 130 and separated from plasma formation zone 135 by a layer of dielectric material 147) defines the beginning of plasma formation zone 135 and spacers 136 define its extent. Gas enters applicator head 130 through opening 141 and an electric contact from electrode 145 is provided via opening 142.

FIGS. 5A-5D are high level schematic illustrations of film cross sections, according to some embodiments of the invention. FIG. 5A schematically illustrates film 110 of biocompatible material 92 with attached adhesive tape 115 at edges 111. FIG. 5B schematically illustrates film 110 with thickened reinforcement zones 152. FIG. 5C schematically illustrates film 110 with pores 151 going through material 92 to enable drainage of fluid. FIG. 5D schematically illustrates film 110 with hollows or bubbles 153 to enable drainage of fluid and make plasma welding more effective, as described above.

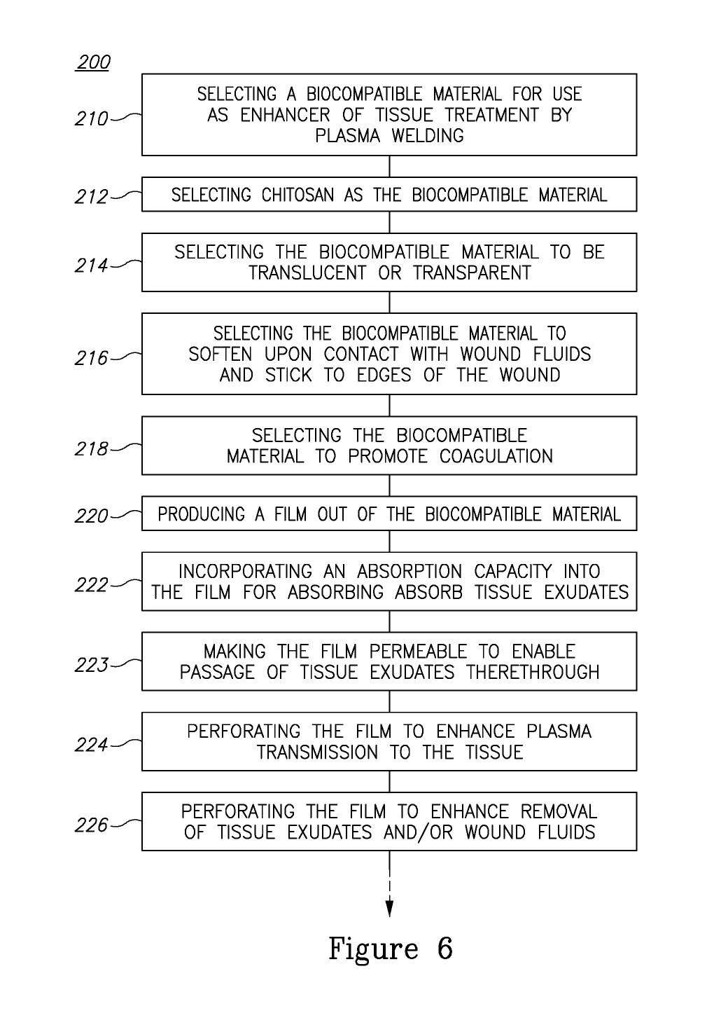

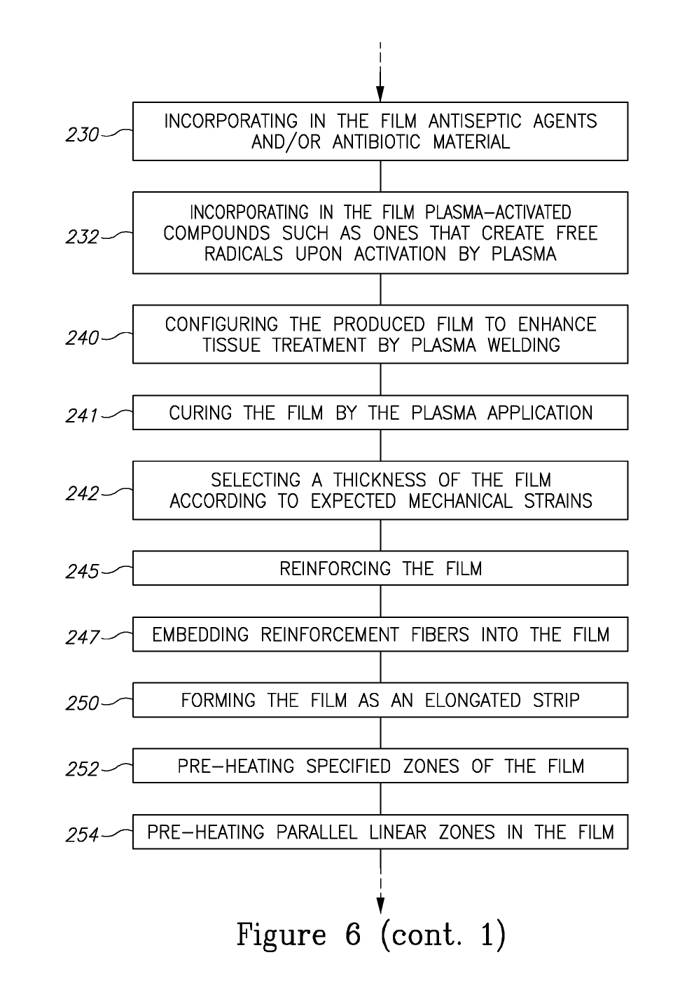

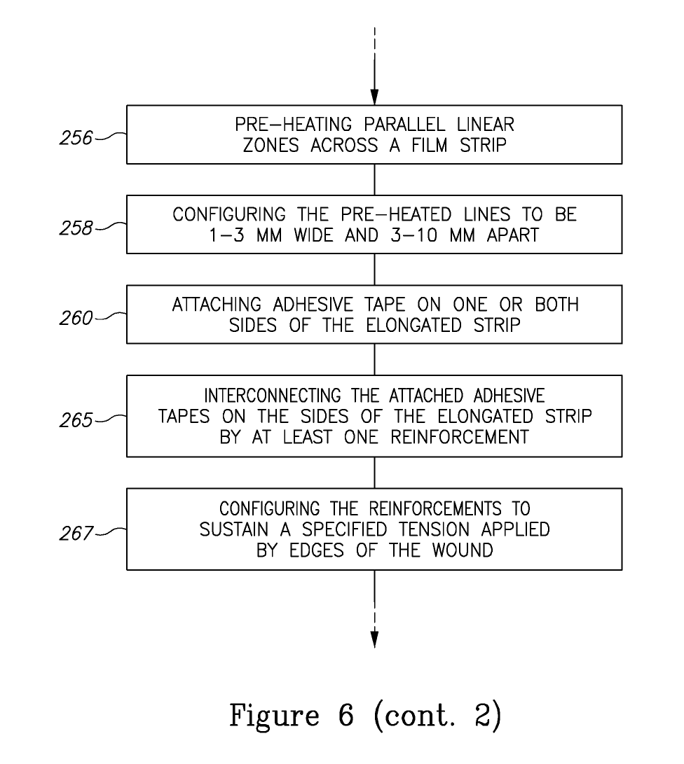

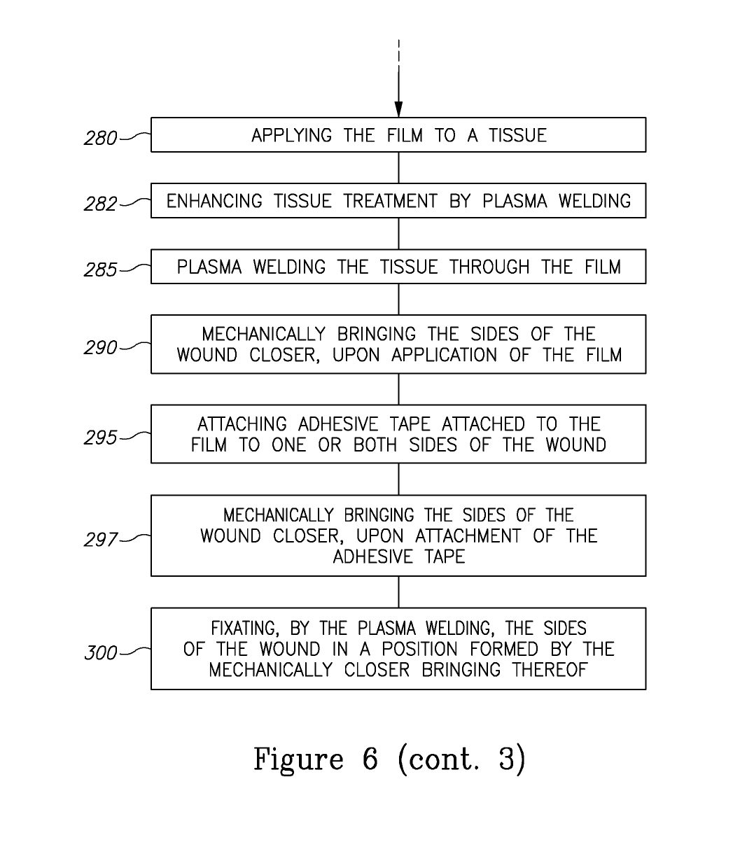

FIG. 6 is a high level schematic illustration of a method 200, according to some embodiments of the invention. Method 200 comprises producing film 110 and/or using film 110 or kit 100 to treat tissue, wounds and organs, and comprises any of the following stages.

Method 200 may comprise selecting a biocompatible material for use as enhancer of tissue treatment by plasma welding (stage 210) and producing a film out of the biocompatible material (stage 220), comprising configuring the produced film to enhance tissue treatment by plasma welding (stage 230). Welded tissue may comprise any type of tissue, internal or external, including lesions such as cuts and wound, and internal or external organs.

In certain embodiments, configuring 230 may comprise forming the film as an elongated strip (stage 250) and producing 220 may further comprise attaching adhesive tape on at least one long side or on both long sides of the elongated strip (stage 260) and optionally interconnecting attached adhesive tapes on the sides of the elongated strip by at least one reinforcement (stage 265). The at least one reinforcement may be configured to sustain a specified tension applied by edges of the wound (stage 267). In certain embodiments, method 200 comprises reinforcing the film (stage 245), e.g., by embedding reinforcement fibers into the film (stage 247). Reinforcing the film (stage 245) may comprise pre-heating at least one specified zone of the film (stage 252) such as a plurality of parallel linear zones (stage 254), optionally across the film strip (stage 256), the linear pre-heated zones traversing a narrow dimension of the strip. For example, the parallel linear zones may be configured to be 1-3 mm wide and are 3-10 mm apart (stage 258).

In certain embodiments, method 200 may further comprise curing the film by the plasma application (stage 241) to mechanically stabilize the treated area. The curing may enhance adhesion to the tissue and resistance to fluids, and determine the degree of permeability of the welded film.

In certain embodiments, producing the film 220 may further comprise selecting a thickness of the film according to expected mechanical strains (stage 242).

Method 200 may comprise any of the following stages: selecting chitosan as the biocompatible material (stage 212), selecting the biocompatible material to be translucent or transparent (stage 214), selecting the biocompatible material to soften upon contact with tissue exudates such as wound fluid and stick to edges of the wound (stage 216) and selecting the biocompatible material to promote coagulation (stage 218), as explained above.

In certain embodiments, producing 220 further comprises incorporating an absorption capacity into the film, configured to absorb wound fluid or tissue exudates (stage 222). Method 200 may further comprise making the film permeable to enable passage of tissue exudates therethrough (stage 223). Producing the film 220 may further comprise perforating the film to enhance plasma transmission to the tissue (stage 224) and/or removal of wound fluid or tissue exudates (stage 226).

Producing the film 220 may further comprise incorporating in the film at least one of an antiseptic agent and an antibiotic material (stage 230). Producing the film 220 may further comprise incorporating in the film at least one plasma-activated compound such as one selected to create free radicals upon activation by plasma (stage 232).

In certain embodiments, method 200 may comprise tissue treatment by applying to a tissue a film made of biocompatible material selected to enhance tissue treatment by plasma welding (stage 280) and plasma welding the tissue through the film (stage 285) and/or enhancing tissue treatment by plasma welding (stage 282).

Method 200 may further comprise mechanically bringing the sides of the wound closer upon application of the film (stage 290) and fixating, by the plasma welding, the sides of the wound in a position formed by the mechanically closer bringing thereof (stage 300).

In certain embodiments, e.g., when the film is formed as an elongated strip comprising attached adhesive tape on at least one long side of the elongated strip, applying the film 280 may further comprise attaching the adhesive tape to one or both sides of the wound (stage 295) and mechanically bringing the sides of the wound closer upon attaching the adhesive tape (stage 297).

In an improved embodiment of exemplary FIG. 15 of PCT/IL2012/050162, it is provided a plasma treatment device that comprises an applicator head having an end configured to contact a treatment surface, the applicator head being configured for connection to a gas source and to a source of energy in order to enable energy to activate the gas and form a plasma; at least one spacer configured for location on a distal end of the applicator head, the at least one spacer being further configured to contact a treatment surface and being sized to maintain the plasma at least 2 mm from the treatment surface when the at least one spacer is in contact with the treatment surface; and at least one vent region associated with the at least one spacer, the at least one vent region begin configured to permit gas entering the spacer to escape.

Optionally, the at least one spacer is detachable from the applicator head. Optionally, the at least one spacer includes a plurality of spaced-apart stand-off legs. Optionally, the at least one spacer includes a tube having openings that act as vents. It should be mentioned that the tube can have a circular profile as well as any other profile in any of the embodiments shown herein. Optionally, the at least one spacer is integrally connected to the applicator head. Optionally, the at least one vent region and the at least one spacer are configured to enable a positive pressure to be maintained within an area bounded by the at least one spacer when gas flows into the area. The positive pressure is maintained by keeping the venting area's surface less than the entrance area. As an example, if the Input gas conduct surface is 3 mm2, then the total vent area should be less than this. This is similar idea as in the “Shower Head” at the end of the document. Optionally, the surface area of the vent region is less than the surface area of the gas inlet. Optionally, the at least one spacer is configured to maintain plasma at a distance of at least between 4 mm and 8 mm from the treatment surface.

It is provided also a plasma treatment device that comprises an applicator head having an end configured to contact an elongated treatment zone on a treatment surface, the applicator head being configured for connection to a gas source and to a source of energy in order to enable energy to activate the gas and form a plasma; a spacer structure configured for location on a distal end of the applicator head, the spacer structure defining opposing openings on opposite lateral sides of the applicator head with an unobstructed working axis therebetween, and wherein the spacer structure is configured such that when held against the treatment surface with the working axis aligned with the elongated treatment zone, the applicator head and the spacer structure may be slid in the direction of the working axis without contacting the treatment zone.

Optionally, at least a portion of the spacer structure is transparent or translucent in order to enable viewing of treatment zone through the spacer structure. Optionally, the device further comprises a plasma zone within one or more of the spacer structure and the applicator head, wherein the applicator head and spacer structure are configured to permit energy to radiate from the plasma zone to the treatment zone as the applicator head and spacer structure are slid along the treatment surface in non-contacting relation to the treatment zone. Optionally, the spacer structure is detachably connected to the application head. Optionally, the spacer structure includes at least two opposing wall sections. Optionally, the spacer is configured to maintain plasma at a distance of at least between 4 mm and 8 mm from the treatment surface, and should be 1 mm to 8 mm preferably.

Another embodiment of the invention provides a device for reconnecting severed tissue, the device comprises: an applicator head having a tissue engaging end configured to contact a skin surface containing a severed tissue area, the tissue engaging end having an elongated opening therein, the elongated opening having a length and a width, the length being at least one and a half times the width; a plasma formation zone in the applicator head, the plasma formation zone being configured such that when the head is pressed against the skin surface the zone lies above the skin surface; at least one energy emitter integrated with the head; at least one gas conduit having at least one opening integrated with the head and configured for conveying gas to the zone, wherein the at least one opening and the at least one energy emitter are arranged to enable a cold plasma to form along a majority of the elongated opening when the at least one energy emitter delivers energy to the zone and gas flows through the at least one opening.

Optionally, the elongated opening has a length of between 3 cm and 9 cm. Optionally, the energy emitter is a band that substantially surrounds the elongated opening. Optionally, the energy emitter band includes a coil. Optionally, the device further includes a spacer for maintaining plasma a distance from the treatment surface, and wherein the spacer contains lateral open ends for minimizing contact between the spacer and the severed tissue as the applicator head is moved laterally across a lacerated region of severed tissue. Optionally, the device further includes at least one second gas conduit having at least one opening integrated with the head and configured for conveying gas away from the zone. Optionally, the at least one first gas conduit and the at least one second gas conduit are configured to maintain a positive pressure of the gas in the zone. Optionally, the at least one opening and the at least one energy emitter are arranged to enable a cold plasma to form along substantially an entire length of the elongated opening. Optionally, the opening structured is made of a plurality of small non elongated openings that form an elongated opening.

It was seen that the film used for welding tissues becomes flexible and elastic when it comes in contact with the patient's blood. This change in the physical properties of the film makes the application of the Chitoplast difficult and the incision edged approximation is compromised. Additionally, the strength of the film is significantly lower when moisture, a phenomenon that imposes delicate and careful handling of the film prior of welding it. Heating lines are used to improve the physical properties of the film. The material properties such as tensile strength and elasticity are improved when the film comes in contact with the patient's blood. Therefore, it is provided in accordance with a preferred embodiment of the present invention, an adhesive wound closure that comprises: a sheet of solder film; a first elongated band of adhesive on a first side of the sheet; a second elongated band of adhesive on a second side of the sheet; wherein the first and second adhesive bands bound an intermediate non-adhesive band of the solder material and wherein a composite structure of the first band, the second band, and the intermediate non-adhesive band of solder material have a tensile strength chosen so that when the first elongated band of adhesive is adhered along one elongated edge of a tissue incision, and the second elongated band is adhered along a second elongated edge of a tissue incision, the composite structure holds opposing incision edges adjacent each other with the non-adhesive intermediate band overlying the opposing incision edges.

It should be noted that the solder film is configured to allow plasma passage through it and interact with the incision below it. The solder film can comprise chitosan, fibrin, and/or other natural or synthetic film. Optionally, the solder film. Optionally, the first and second adhesive bands include adhesive overlying portions of the sheet of solder material. Optionally, the adhesive wound closure further includes a first carrier band and a second carrier band connected to opposing edges of the sheet of the solder material, and wherein the first elongated band of adhesive is located on the first carrier band and the second elongated band of adhesive is located on the second carrier band. Optionally, the solder material includes chitosan. Optionally, the solder material includes a chitosan film. Optionally, the solder material includes a chitosan film with heating lines on it. The heating lines across the sheet of solder material are important to improve the physical properties of the sheet. Optionally, the heating lines are in the width between 1-3 mm Optionally, the distance of two proximate heating lines is between 3-10 mm Optionally, the solder material is translucent thereby enabling viewing of the opposing incision edges therethrough. Optionally, the composite structure has a length of at least 7 cm. Optionally, the composite structure has a length of at least 12 cm. Optionally, the composite structure has a length of between 7 cm and 20 cm. Optionally, the heating lines are across the solder film. Optionally, the heating lines are lengthwise and crosswise. Optionally, the intermediate non-adhesive band of the solder material is configured to disintegrate over time. Optionally, the first adhesive strip is spaced at least 3 mm from the second adhesive strip. Optionally, the first and second adhesive strips further include a base sheet and the adhesive strip is located on one face of the base sheet. Optionally, the first and second adhesive strips are selected from the group consisting of Polyester Nonwoven Medical that is coated with hypoallergenic, pressure sensitive acrylate adhesive and covered with a silicon liner until application to the skin. Chitosan plaster with heating lines on it substantially in accordance with a preferred embodiment of the present invention.

A method is disclosed of connecting disconnected tissue, the method comprises: applying a mechanical closure across opposing edges of the disconnected tissue to maintain the opposing edges in proximity to each other; while the mechanical closure is in place, exposing the opposing edges to cold plasma about 40° C., non-charring plasma); maintaining the mechanical closure across the opposing edges for a period following the exposure to cold plasma.

Optionally, the mechanical closure includes a tissue engaging surface containing chitosan. Optionally, the mechanical closure includes a strip of chitosan film configured to overly opposing edges of tissue to be joined. Optionally, the mechanical closure includes an elongated band of chitosan film sandwiched on opposing lateral sides by first and second elongated bands of adhesive. Optionally, the mechanical closure includes a removable or degradable suture or staple. Optionally, the disconnected tissue includes opposing edges of a cesarean-section incision. Optionally, the disconnected tissue includes opposing edges of a cesarean-section incision, and wherein the mechanical closure includes at least one elongated adhesive strip of at least 10 cm in length.

It is provided a wound closure kit, comprising: at least one composite strip including a band of solder film sandwiched on opposing lateral sides by first and second elongated bands of adhesive, the composite strip being configured such that when each of the first and second elongated bands of adhesive are applied on opposing edges of disconnected tissue, the band of solder film overlies the opposing edges; and a cold plasma applicator head having a plasma opening on a distal end thereof, the applicator head being configured to apply energy from a plasma to the disconnected tissue edges through the band of solder material.

Optionally, the solder material of the film is selected from the group comprising chitosan, fibrin, and other a natural or synthetic blood clotting agents. Optionally, the solder material includes chitosan. Optionally, the solder material includes a chitosan film. Optionally, the solder material band is translucent or transparent thereby enabling viewing of the opposing tissue edges therethrough. Optionally, the composite strip includes two spaced-apart bands of adhesive on a sheet of the solder material.

A device for reconnecting severed tissue is provided wherein the device comprises: an applicator head having an end configured to contact a skin surface containing a severed tissue area, the head defining a plasma formation zone, such that when the head is pressed against the skin surface, the plasma formation zone lies above the skin surface; at least one radio frequency energy emitter integrated with the head, including at least one electrode spaced by a streamer-free dielectric from the plasma formation zone; at least one gas conduit having at least one opening integrated with the head and configured to convey gas to the zone, wherein the at least one opening and the at least one energy emitter are arranged to enable a cold plasma that is substantially free of streamers to form in the zone when the at least one energy emitter delivers energy to the zone and gas flows through the at least one opening.

Optionally, the energy emitter includes an RF electrode. Optionally, the energy emitter includes an electrode and a glass-free dielectric barrier separating the electrode from the zone. Optionally, the plastic material is the dielectric tube material, the material that separates between the RF and the gas is selected from the group consisting of Polycarbonate, Polyurethane, Acrylonitrile butadiene styrene (ABS) etc. Optionally, the dielectric material is at least 0.1 mm thick. Optionally, the dielectric material is between 0.1 mm and 3 mm thick. The device may be configured to have a separation radius between streamers of not more than 2 mm at the application point—meaning that the streamers are spread uniformly and densely making it a uniform plasma and essentially streamer free plasma. Optionally, the device further comprises a dielectric material separating the at least one energy emitter from the zone.

There is also provided in accordance with another preferred embodiment of the present invention, a plasma treatment device that comprises: at least one processor configured to control plasma formation in a plasma applicator head having at least one energy emitter, a plasma formation zone, and a gas conduit through which gas flows to the plasma formation zone, the at least one processor further configured control operating conditions of the plasma formation such that: gas flux across the plasma formation zone is between 0.05 and 0.4 liters/min•mm²; a duty cycle of the power supplied to the energy emitter is between 2.5% and 15%; and a carrier frequency of the energy emitter is between 0.5 MHz and 5 MHz, a RF voltage of the power supplied to the energy emitter between 2.5 kV and 7 kV. Optionally, the energy emitter is an RF electrode. Optionally, the gas flux is about 0.2 liters/min•mm² Optionally, the duty cycle is about 5%. Optionally, the carrier frequency is about 2 MHz. Optionally, the foregoing parameters are adjusted to provide a cold plasma with a density and temperature suitable for tissue welding. Optionally, the plasma treatment device includes a plasma applicator head having at least one energy emitter, a plasma formation zone, and a gas conduit through which gas flows to the plasma formation zone.

A plasma treatment device is provided that comprises: at least one processor configured to control plasma formation in a plasma applicator head having at least one energy emitter, a plasma formation zone, and a gas conduit through which gas flows to the plasma formation zone, the at least one processor further configured to cause RF energy to be delivered to the energy emitter in spaced apart peaks, and wherein the at least one processor is configured to cause peaks to occur during less than 20% of a tissue welding procedure, and wherein each peak corresponds to a voltage greater than 3 kV.

Optionally, the energy emitter is an RF electrode. Optionally, the peaks occur between about 3% and about 15% of the tissue welding procedure. Optionally, the peaks occur between 5% and 10% of tissue welding procedure. Optionally, the at least one processor is configured to modulate a duty cycle in response to feedback received from the plasma applicator head, and wherein the feedback includes information about conductivity, resistance, capacitance, impedance, density, distance to the treated area and/or temperature of the cold plasma. Optionally, the at least one processor is configured to modulate a rate of gas flow to the plasma formation zone. Optionally, the at least one first processor is configured to modulate a duty cycle based on the gas flow rate in the plasma formation zone. Optionally, the energy emitter is an RF electrode and wherein the at least one processor is configured to modulate the RF carrier frequency. Optionally, the carrier frequency is about 2 MHz. Optionally, the at least one processor is configured to modulate a duty cycle based on the carrier frequency of the energy emitter. Optionally, the at least one processor is configured to modulate a duty cycle based on the plasma distance from the treatment surface.

In accordance with another embodiment, gas flow shutter for flow determination in each tip. A tube (or other shaped passage) in the way of the gas to the plasma formation zone is provided, where the passage of the gas is confined to a specific flow in a specific pressure that is adjusted using a thin tube that is located in the gas entrance to the tip. This enables working with one input pressure but different gas flows for different plasma tips. This saves the need for an expensive gas flow controller (MFC).

A “recipe” of plasma parameters where one of the parameters is a defined duration of welding. This duration defines the plasma dosage for the specific welding segment. After the time has passed, the plasma shuts off, there is a pre-defined waiting time where the plasma can't be ignited again and then, upon pressing the button, the plasma is ignited again for welding of the next segment. (This option is mainly for the WideTip).

A simple conductive rod (metal or plastic covered with metal) that is structured in a way that will fit the plasma tip exactly to ignite the plasma at first ignition (where its ignition is difficult). The rod is configured to reach the plasma inner tube where the RF exciter is located from the outside (the best location to ignite the plasma). The rod has “stoppers” that let it be inserted perfectly to the right location and not pass it.

Optionally, a shower head is provided that is configured to maintain positive pressure in the side of the gas entrance and by that distributing the gas uniformly (lower surface of holes than gas entrance surface). A plate with holes that enables the gas to pass uniformly and be distributed to the plasma region. In order to receive good distribution, the “shower head” need to be configured to maintain positive pressure in the proximal side (close to the gas source). This is achieved by having the total surface of the holes in the shower head smaller than the surface of the gas entrance conduit. The equation is: R₁/n^{1/2}>R₂, where: R1—Gas conduct radius, n—Shower head number of holes, R2—Shower head holes radius.

In order to overcome the above mentioned problem of moisture, reinforcement of the BioWedling film is needed. One option to solve the problem is a series of lines (“bridges”) that are connected to the adhesive plasters from both sides and add mechanical strength. Shown previously herewith. The reinforcement can be made of a different material as a medical plaster (i.e.—Steristrip), synthetic fibers or the chitosan itself but after “heating”. Heating lines are used to improve the physical properties of the material, properties such as tensile strength and elasticity when the film comes in contact with the patient's blood. Optionally, the reinforcement lines are in the width of between 1-3 mm. Optionally, the distance between two proximate heating lines is between 3-10 mm.

Certain embodiments comprise a plasma treatment device, comprising: an applicator head having an end configured to contact a treatment surface, the applicator head being configured for connection to a gas source and to a source of energy in order to enable energy to activate the gas and form a plasma; a spacer configured for location on a distal end of the applicator head, the spacer being further configured to contact a treatment surface and being sized to maintain the plasma at least 3 mm from the treatment surface when the spacer is in contact with the treatment surface; and at least one vent region associated with the spacer, the at least one vent region begin configured to permit gas entering the spacer to escape.

In certain embodiments at least one of the following occurs: the spacer is detachable from the applicator head; the spacer is integrally connected to the applicator head; the at least one vent region and the spacer are configured to enable a positive pressure to be maintained within the spacer when gas flows into the spacer; the spacer is configured to maintain plasma at a distance of at least between 4 mm and 8 mm from the treatment surface.

Certain embodiments comprise a plasma treatment device, comprising: an applicator head; a plasma formation zone associated with the applicator head; a gas conduit for delivering gas to the plasma formation zone; and an radio frequency exciter band substantially surrounding a periphery of the plasma formation zone, the exciter band being configured to ignite a plasma in the plasma formation zone when gas is delivered to the plasma formation zone via the conduit.

In certain embodiments at least one of the following occurs: the exciter band is a wire coil; the exciter band is a metal ring; the device further comprises a dielectric material substantially separating the exciter band from the plasma formation zone; the exciter band is located proximate an opening of the plasma formation zone; the zone has an elongated shape with a length at least four times its width; the exciter band is configured to substantially uniformly deliver energy to the gas, to thereby cause a uniform plasma region, substantially free of streamers.

Certain embodiments comprise a plasma treatment device, comprising: an applicator head having an end configured to contact an elongated treatment zone on a treatment surface, the applicator head being configured for connection to a gas source and to a source of energy in order to enable energy to activate the gas and form a plasma; a spacer structure configured for location on a distal end of the applicator head, the spacer structure defining opposing openings on opposite lateral sides of the applicator head with an unobstructed working axis therebetween, and wherein the spacer structure is configured such that when held against the treatment surface with the working axis aligned with the elongated treatment zone, the applicator head and the spacer structure may be slid in the direction of the working axis without contacting the treatment zone.

In certain embodiments at least one of the following occurs: at least a portion of the spacer structure is translucent in order to enable viewing of treatment zone through the spacer structure; the device further comprises a plasma zone within one or more of the spacer structure and the applicator head, wherein the applicator head and spacer structure are configured to permit energy to radiate from the plasma zone to the treatment zone as the applicator head and spacer structure are slid along the treatment surface in non-contacting relation to the treatment zone; the spacer structure is detachably connected to the application head; the spacer structure includes at least two opposing wall sections; the spacer is configured to maintain plasma at a distance of at least between 4 mm and 8 mm from the treatment surface.

Certain embodiments comprise a device for reconnecting severed tissue, the device comprising: an applicator head having a tissue engaging end configured to contact a skin surface containing a severed tissue area, the tissue engaging end having an elongated opening therein, the elongated opening having a length and a width, the length being at least four times the width; a plasma formation zone in the applicator head, the plasma formation zone being configured such that when the head is pressed against the skin surface the zone lies above the skin surface; at least one energy emitter integrated with the head; at least one gas conduit having at least one opening integrated with the head and configured for conveying gas to the zone, wherein the at least one opening and the at least one energy emitter are arranged to enable a cold plasma to form along a majority of the elongated opening when the at least one energy emitter delivers energy to the zone and gas flows through the at least one opening.

In certain embodiments at least one of the following occurs: the elongated opening has a length of between 3 cm and 9 cm; the energy emitter is a band that substantially surrounds the elongated opening; the device further comprises a spacer for maintaining plasma a distance from the treatment surface, and wherein the spacer contains lateral open ends for minimizing contact between the spacer and the severed tissue as the applicator head is moved laterally across a lacerated region of severed tissue; the device further comprises at least one second gas conduit having at least one opening integrated with the head and configured for conveying gas away from the zone; the at least one first gas conduit and the at least one second gas conduit are configured to maintain a positive pressure of the gas in the zone; the at least one opening and the at least one energy emitter are arranged to enable a cold plasma to form along substantially an entire length of the elongated opening.

Certain embodiments comprise an adhesive wound closure, comprising: a first elongated band of adhesive; a second elongated band of adhesive; and a scabbing material band in between and interconnecting the first band and the second band, wherein a composite structure of the first band, the second band, and the scabbing material band have a tensile strength chosen so that when the first elongated band of adhesive is adhered along one elongated edge of a tissue incision, and the second elongated band is adhered along a second elongated edge of a tissue incision, the composite structure holds opposing incision edges adjacent each other with the scabbing material overlying the opposing incision edges.

In certain embodiments at least one of the following occurs: the scabbing material is selected from the group comprising chitosan, fibrin, and other natural or synthetic blood clotting agents; the scabbing material band includes chitosan; the scabbing material band includes a chitosan film; the scabbing material band is translucent thereby enabling viewing of the opposing incision edges therethrough; the composite structure has a length of at least 7 cm; the composite structure has a length of at least 12 cm; the composite structure has a length of between 7 cm and 16 cm; the scabbing material is configured to absorb into the incision following application of cold plasma; the first adhesive strip is spaced between 1 mm and 30 mm from the second adhesive strip.

Certain embodiments comprise a method of connecting disconnected tissue, the method comprising: applying a mechanical closure across opposing edges of the disconnected tissue to maintain the opposing edges in proximity to each other; while the mechanical closure is in place, exposing the opposing edges to cold plasma; and maintaining the mechanical closure across the opposing edges for a period following the exposure to cold plasma.

In certain embodiments at least one of the following occurs: the mechanical closure includes a tissue engaging surface containing chitosan; the mechanical closure includes a strip of chitosan film configured to overly opposing edges of tissue to be joined; the mechanical closure includes an elongated band of chitosan film sandwiched on opposing lateral sides by first and second elongated bands of adhesive; the mechanical closure includes a removable or degradable suture or staple; the disconnected tissue includes opposing edges of a cesarean-section incision; the disconnected tissue includes opposing edges of a cesarean-section incision, and wherein the mechanical closure includes at least one elongated adhesive strip of at least 10 cm in length.

Certain embodiments comprise a wound closure kit, comprising: at least one composite strip including a band of scabbing material sandwiched on opposing lateral sides by first and second elongated bands of adhesive, the composite strip being configured such that when each of the first and second elongated bands of adhesive are applied on opposing edges of disconnected tissue, the band of scabbing material overlies the opposing edges; and a cold plasma applicator head having a plasma opening on a distal end thereof, the applicator head being configured to apply energy from a plasma to the disconnected tissue edges through the band of scabbing material.

In certain embodiments at least one of the following occurs: the scabbing material is selected from the group comprising chitosan, fibrin, and other a natural or synthetic blood clotting agents; the scabbing material includes chitosan; the scabbing material includes a chitosan film; the scabbing material band is translucent thereby enabling viewing of the opposing tissue edges therethrough.

Certain embodiments comprise a device for reconnecting severed tissue, the device comprising: an applicator head having an end configured to contact a skin surface containing a severed tissue area, the head defining a plasma formation zone, such that when the head is pressed against the skin surface, the plasma formation zone lies above the skin surface; at least one radio frequency energy emitter integrated with the head; at least one gas conduit having at least one opening integrated with the head and configured to convey gas to the zone, wherein the at least one opening and the at least one energy emitter are arranged to enable a cold plasma that is substantially free of streamers to form in the zone when the at least one energy emitter delivers energy to the zone and gas flows through the at least one opening.

In certain embodiments at least one of the following occurs: the energy emitter is an RF electrode; the device is configured to have less than 5 streamers per minute under typical operating conditions; the device further comprises a dielectric material separating the at least one energy emitter from the zone.

Certain embodiments comprise a plasma treatment device, comprising: at least one processor configured to control plasma formation in a plasma applicator head having at least one energy emitter, a plasma formation zone, and a gas conduit through which gas flows to the plasma formation zone, the at least one processor further configured control operating conditions of the plasma formation such that: gas flux across the plasma formation zone is between 0.1 and 0.4 liters/mm2; a duty cycle of the power supplied to the energy emitter is between 3% and 15%; and a carrier frequency of the energy emitter is between 0.5 MHz and 5 MHz.

In certain embodiments at least one of the following occurs: the energy emitter is an RF electrode; the gas flux is about 0.2 liters/mm2; the duty cycle is about 5%; the carrier frequency is about 2 MHz; the foregoing parameters are adjusted to provide a cold plasma with a density and temperature suitable for tissue welding; the plasma treatment device includes a plasma applicator head having at least one energy emitter, a plasma formation zone, and a gas conduit through which gas flows to the plasma formation zone.

Certain embodiments comprise a plasma treatment device, comprising: at least one processor configured to control plasma formation in a plasma applicator head having at least one energy emitter, a plasma formation zone, and a gas conduit through which gas flows to the plasma formation zone, the at least one processor further configured to cause RF energy to be delivered to the energy emitter in spaced apart peaks, and wherein the at least one processor is configured to cause peaks to occur during less than 20% of a tissue welding procedure, and wherein each peak corresponds to a voltage greater than 10% of a maximal voltage.

In certain embodiments at least one of the following occurs: the energy emitter is an RF electrode; the peaks occur between about 3% and about 15% of the tissue welding procedure; the peaks occur between 5% and 10% of tissue welding procedure; the at least one processor is configured to modulate a duty cycle in response to feedback received from the plasma applicator head, and wherein the feedback includes information about conductivity, resistance, capacitance, impedance, density and/or temperature of the cold plasma; the at least one processor is configured to modulate a rate of gas flow to the plasma formation zone; the at least one first processor is configured to modulate a duty cycle based on the gas flow rate in the plasma formation zone; the energy emitter is an RF electrode and wherein the at least one processor is configured to modulate the RF carrier frequency; the carrier frequency is about 2 MHz; the at least one processor is configured to modulate a duty cycle based on the carrier frequency of the energy emitter.

In the above description, an embodiment is an example or implementation of the invention. The various appearances of “one embodiment”, “an embodiment”, “certain embodiments” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Certain embodiments of the invention may include features from different embodiments disclosed above, and certain embodiments may incorporate elements from other embodiments disclosed above. The disclosure of elements of the invention in the context of a specific embodiment is not to be taken as limiting their used in the specific embodiment alone.

Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A film made of biocompatible material selected to enhance tissue treatment by plasma welding, wherein the film is formed as an elongated strip and further comprising attached adhesive tape on at least one long side of the elongated strip, the film further comprising at least one reinforced zone of pre-heated film material.

2. The film of claim 1, further comprising attached adhesive tape on both long sides of the elongated strip and further comprising at least one reinforcement interconnecting the attached adhesive tapes on the sides of the elongated strip.

3. (canceled)

4. The film of claim 1, wherein the at least one reinforced zone further comprises a plurality of linear reinforced zones traversing a narrow dimension of the strip, wherein the linear reinforced zones are 1-3 mm wide and are 3-10 mm apart.

5. (canceled)

6. (canceled)

7. The film of claim 1, wherein the biocompatible material is selected to soften upon contact with tissue exudates and stick to the tissue and wherein the film is perforated to enhance at least one of plasma transmission to the tissue and removal of tissue fluid.

8-13. (canceled)

14. The film of claim 1, further comprising at least one plasma-activated compound, selected to create free radicals upon activation by plasma.

15. (canceled)

16. A kit comprising:
a film made of biocompatible material selected to enhance tissue treatment by plasma welding; and
an applicator head arranged to connect to a plasma generating device and to plasma-treat a tissue,
wherein the film is formed as an elongated strip having a specified width and the film further comprises attached adhesive tape on both long sides thereof.

17. The kit of claim 16, wherein the applicator head has a width smaller than the specified width of the film strip.

18. The kit of claim 17, wherein the applicator head is at least three times longer than wide.

19. The kit of claim 16, wherein the applicator head comprises spacers configured to optimize plasma welding of the tissue and the film.

20. The kit of claim 19, wherein the spacers are 3-9 mm apart and maintain a distance of between 4-8 mm between the plasma and the film.

21. The kit of claim 16, wherein the applicator head comprises plastic dielectric material between an electrode and a plasma formation zone enclosed by the applicator head.

22. The kit of claim 21, wherein the plastic dielectric material is 0.1-3 mm thick.

23. The kit of claim 16, wherein the film and the applicator head are designed to operate under a gas flux,. across a plasma formation zone enclosed by the applicator head, which is between 0.05 and 0.4 liters/min•mm², and, with respect to power supplied to an energy emitter in the plasma generating device, to have a duty cycle between 2.5% and 15%, a carrier frequency between 0.5 MHz and 5 MHz, and a RF voltage 2.5 kV and 7 kV.

24. The kit of claim 16, wherein the film is arranged to enhance wound treatment by plasma welding and wherein the film is reinforced across its width by at least one of: a reinforcement member interconnecting the attached adhesive tapes on the sides of the elongated strip, a reinforced zone of pre-heated film material, a thickened film zone, and embedded reinforcement fibers.

25. The kit of claim 16, wherein the film is arranged to enhance wound treatment by plasma welding and is perforated to enhance at least one of plasma transmission to the wound and removal of wound fluid, and wherein the biocompatible material is selected to soften upon plasma application and stick to edges of the wound.

26. (canceled)

27. (canceled)

28. A method comprising
selecting a biocompatible material for use as enhancer of wound treatment by plasma welding; and
producing a film out of the biocompatible material, the producing comprises configuring the produced film to enhance wound treatment by plasma welding and reinforcing the film,
wherein the configuring comprises forming the film as an elongated strip and further comprising attaching adhesive tape on at least one long side of the elongated strip.

29. The method of claim 28, further comprising attaching adhesive tape on both long sides of the elongated strip and interconnecting the attached adhesive tapes on the sides of the elongated strip by at least one reinforcement member.

30. (canceled)

31. The method of claim 29, wherein the film is arranged to enhance wound treatment by plasma welding and the method further comprises configuring the at least one reinforcement member to sustain a specified tension applied by edges of the wound.

32. (canceled)

33. The method of claim 28, wherein the reinforcing comprises pre-heating at least one specified zone of the film.

34. The method of claim 33, wherein the at least one pre-heated zone comprises a plurality of parallel linear zones and wherein the configuring comprises forming the film as an elongated strip and the linear pre-heated zones traverse a narrow dimension of the strip.

35-113. (canceled)
